(19) United States
(12) Patent Application Publication
(10) Pub. No.: US 2010/0053209 A1
(43) Pub. Date: Mar. 4, 2010

(54) System for Processing Medical Image data to Provide Vascular Function Information

(75) Inventors: John Christopher Rauch, Warwick, RI (US); John Baumgart, Hoffman Estates, IL (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc., Malvern, PA (US)

(21) Appl. No.: 12/550,719

(22) Filed: Aug. 31, 2009

Related U.S. Application Data
(60) Provisional application No. 61/093,002, filed on Aug. 29, 2008.
(60) Provisional application No. 61/092,997, filed on Aug. 29, 2008.

(51) Int. Cl. G09G 5/00; A61B 6/00

(52) U.S. Cl. 345/619; 600/431

(57) ABSTRACT

A system creates a visually (e.g., color) coded 3D image that depicts 3D vascular function information including transit time of blood flow through the anatomy. A system combines 3D medical image data with vessel blood flow information. The system uses at least one repository for storing, 3D image data representing a 3D imaging volume including vessels, in the presence of a contrast agent and 2D image data representing a 2D X-ray image through the imaging volume in the presence of a contrast agent. An image data processor uses the 3D image data and the 2D image data in deriving blood flow related information for the vessels. A display processor provides data representing a composite single displayed image including a vessel structure provided by the 3D image data and the derived blood flow related information.

This is a non-provisional application of provisional application Ser. No. 61/093,002 filed Aug. 29, 2008 and of provisional application Ser. No. 61/092,997 filed Aug. 29, 2008, by J. Baumgart et al.

### FIELD OF THE INVENTION

This invention concerns a system for combining 3D (three dimensional) medical image data with vessel blood flow information and providing a composite single displayed image including a vessel structure provided by the 3D image data and derived blood flow related information.

### BACKGROUND OF THE INVENTION

In diagnosing and treating patients with vascular problems or deficiencies, it is often necessary to examine both the morphologic and functional characteristics of vasculature. Morphologic information includes the size, geometry, number and placement of the vessels in the anatomy. For vascular anatomy, functional information pertains mainly to the flow of blood including transit times, blood flow, and perfusion. In a conventional angiography laboratory, information on vascular morphology and function are typically acquired and reviewed separately. Vascular morphology is accurately appreciated with a 3D (three dimensional) image acquired by a rotational acquisition and reconstructed using computed tomography techniques. Images are acquired with a contrast agent injection to highlight the vessels of interest allowing for direct measurement as well as qualitative evaluation of the individual vessels and entire vasculature. Information about the function of the vasculature is acquired via acquisition and review of digital subtraction angiography (DSA) images derived by subtraction of a mask image containing background detail from a contrast agent enhanced image. If the vessels in question are embedded in soft tissue, ultrasound may also be used to quantify vascular function. A user mentally assimilates and interprets the morphological and functional information from these multiple sources and uses the information in combination to diagnose, plan treatment, or engage in therapeutic activities.

Vascular anatomy can be complex, especially in sick patients, with vessels overlapping, branching, and running in directions perpendicular to standard angiographic viewing orientations. In a DSA image there is no depth information and vessels in the anatomy being imaged appear and disappear as a contrast agent flows through them. However, the process of mentally combining the morphologic and functional information identified in the 3D and DSA (Digital Subtraction Angiography) images requires a physician to correlate multiple overlapped vessels depicted in DSA images with the vasculature presented in a 3D image. The effectiveness of this correlation is dependant on the physician's ability to read a pair of DSA images and infer spatial placement and orientation of the vessels in 3D space. A system according to invention principles addresses these requirements and associated deficiencies and problems.

### SUMMARY OF THE INVENTION

A system generates a visually coded 3D image that depicts both vascular morphology and function by visually (e.g. color) coding functional information (e.g. transit time of blood flow through the anatomy) directly on a 3D morphologic image of the vasculature. The functional information is acquired by iteratively computing and scaling transit time curves for individual voxels and minimizing the difference between transit time curves of pixels in a 2D (two dimensional) image to the calculated transit time curves of corresponding projections through a 3D volume. A system combines 3D medical image data with vessel blood flow information. The system uses at least one repository for storing, 3D image data representing a 3D imaging volume including vessels, in the presence of a contrast agent and 2D image data representing a 2D X-ray image through the imaging volume in the presence of a contrast agent. An image data processor uses the 3D image data and the 2D image data in deriving blood flow related information for the vessels. A display processor provides data representing a composite single displayed image including a vessel structure provided by the 3D image data and the derived blood flow related information.

### BRIEF DESCRIPTION OF THE DRAWING

FIG. 1 shows a system for combining 3D medical image data with vessel blood flow information, according to invention principles.

FIG. 2 shows a DSA image presenting a vessel structure (shown as a grayscale representation of a color coded image), according to invention principles.

FIG. 3 shows a transit time curve for one pixel in the DSA image of FIG. 2, according to invention principles.

FIG. 4 illustrates generation of a composite single transit time curve by taking minimum luminance intensity values from first and second different transit time curves obtained from 3D volume imaging data and 2D images in the volume, according to invention principles.

FIG. 5 illustrates generation of a composite single transit time curve by multiplying and scaling luminance intensity values from first and second different transit time curves obtained from 3D volume imaging data and 2D images in the volume, according to invention principles.

FIGS. 6 and 7 illustrate fitting a Gaussian curve to different portions of a transit time curve, according to invention principles.

FIG. 8 illustrates employing multiple Gaussian curves to approximate a transit time curve, according to invention principles.

FIG. 9 illustrates the projection of a voxel onto two imaging planes to determine the pixels in those planes that are used in computing a transit time curve of a voxel and projection of a single pixel back through the volume to identify the voxels that are evaluated to determine a pixel scaling function according to invention principles.

FIG. 10 shows a flowchart of a process embodiment used by a system for combining 3D medical image data with vessel blood flow information, according to invention principles.

FIG. 11 shows a flowchart of a process embodiment used by a system for combining 3D medical image data with vessel blood flow information, according to invention principles.

### DETAILED DESCRIPTION OF THE INVENTION

A system generates a visually (e.g., color) coded 3D image that depicts 3D vascular function information including transit time of blood flow through the anatomy. A transit time curve identifies blood flow by tracking the flow of contrast agent through a region of the anatomy (tissue or vessel). The transit time curve itself plots the x-ray luminance of a pixel or region of pixels in a DSA sequence over the length of the DSA sequence: the amount of contrast in the region of interest over time. Since the blood is carrying the contrast agent, it is possible to obtain a functional measure of the time required for blood to flow through the vessel by examining the time to peak value or time to leading edge of the transit time curves at different locations in the vessel. The functional information is provided using multiple subtracted angiography acquisitions of patient anatomy, while a 3D image of the vasculature provides the morphology of the vascular anatomy. The functional information for each 3D element, or voxel, is determined by iteratively computing and scaling transit time curves for individual voxels. Individual iterations attempt to minimize a difference between transit time curves of pixels in a 2D image and the calculated transit time curves of corresponding projections through a 3D volume encompassing the 2D image.

The system displays information concerning vascular function in a 3D image by advantageously combining functional and geometric information of the vessels concerned and displaying the information in a single format. The functional information is obtained from digital subtraction angiography images and is overlaid onto a 3D image of the same vasculature. The system automatically merges morphologic and functional information provided by 3D images and angiographic images of vasculature into a single 3D display, enabling a user to view the combined information in a single view and from a user selectable orientation. The automated system enables a user to focus on interpreting the information instead of having to combine it.

A system advantageously depicts DSA images in which blood flow transit time information is displayed with varying colors that identify the time at which blood flow has achieved a desired characteristic. The system computes a transit time curve for each individual pixel in an image or region of interest in an image. A transit time curve identifies luminance intensity of contrast agent detected at a particular pixel location in an image as a function of time and represents blood flow at that pixel in the image. The system is capable of generating a transit time curve for each voxel (a 3D pixel) in a 3D volume. To make use of this information the system generates a 3D image volume colored to depict vascular flow information using the transit time curves computed for each voxel. The voxel transit time curves are computed using the spatial and temporal information provided by multiple DSA image sequences (at least 2) acquired at different imaging orientations.

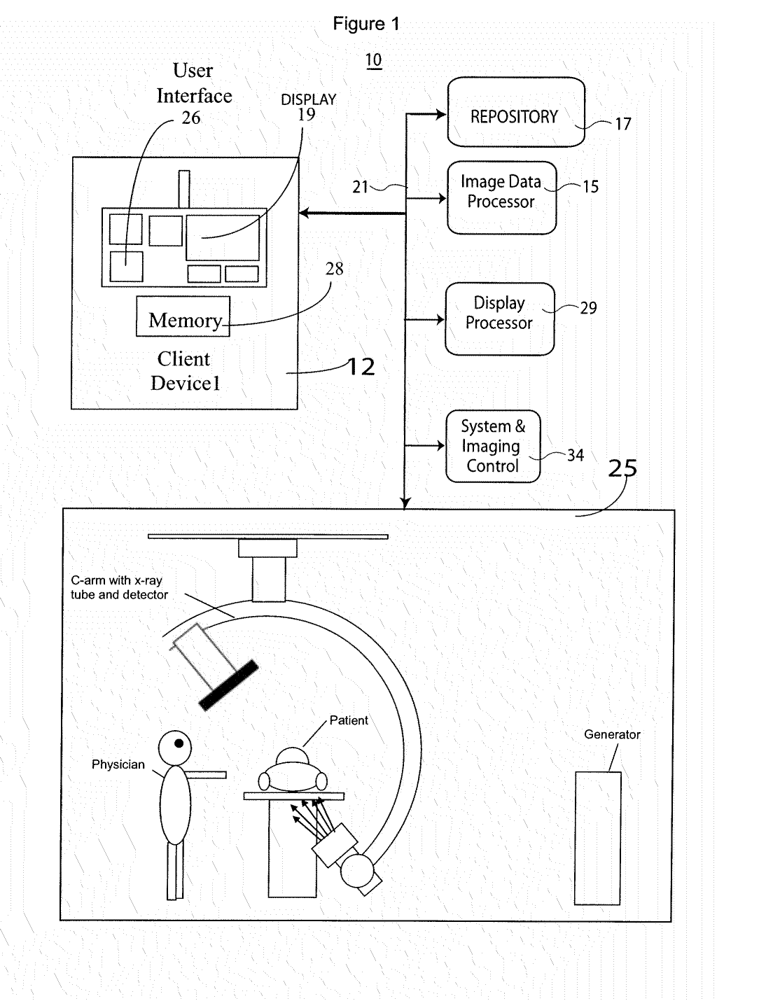

FIG. 1 shows system 10 for combining 3D medical image data with vessel blood flow information. System 10 includes one or more processing devices (e.g., workstations, computers or portable devices such as notebooks, Personal Digital Assistants, phones) 12 that individually include a user interface 26 enabling user interaction with a Graphical User Interface (GUI) and display 19 supporting GUI and medical image presentation in response to predetermined user (e.g., physician) specific preferences. System 10 also includes at least one repository 17, image data processor 15, display processor 29, imaging devices 25 and system and imaging control unit 34. System and imaging control unit 34 controls operation of one or more imaging devices 25 for performing image acquisition of patient anatomy in response to user command. Imaging devices 25 may comprise a single device (e.g., a mono-plane or biplane X-ray imaging system) or multiple imaging devices such as an X-ray imaging system together with a CT scan or Ultrasound system, for example). The units of system 10 intercommunicate via network 21. At least one repository 17 stores medical image studies for patients in DICOM compatible (or other) data format. A medical image study individually includes multiple image series of a patient anatomical portion which in turn individually include multiple images.

One or more imaging devices 25 acquire image data representing a 3D imaging volume of interest of patient anatomy in the presence of a contrast agent and acquire multiple DSA sequential images (which may or may not be synchronized with ECG and respiratory signals) of a vessel structure in the presence of a contrast agent in the 3D volume interest. At least one repository 17 stores 3D image data representing a 3D imaging volume including vessels in the presence of a contrast agent. At least one repository 17 stores 2D image data representing 2D DSA X-ray images through the imaging volume in the presence of a contrast agent. Image data processor 15 uses the 3D image data and the 2D image data in deriving blood flow related information for the vessels. Display processor 19 provides data representing a composite single displayed image including a vessel structure provided by the 3D image data and the derived blood flow related information.

In order to localize the content of two-dimensional (2D) images within a 3D imaging volume acquired by imaging systems 25, at least two separate imaging plane orientations of the same object are used. System 10 generates a 3D image of vasculature with color coded functional information using at least two DSA images acquired by imaging systems 25. As in known 3D image reconstruction methods, the quality of image reconstruction is improved by acquiring additional images at different imaging orientations. System 10 may employ different combinations of multiple monoplane and/or biplane DSA image acquisitions as long as the contrast agent bolus geometry is the same and the DSA image sequences are synchronized to introduction of the contrast agent bolus into patient anatomy. Image data processor 15 adjusts and registers (aligns) a 3D image with 2D DSA images and generates a flow enhanced vascular 3D image. In another embodiment, the process of registering 2D and 3D images may be optional but the process adds flexibility to compensate for movement of the patient or patient support table between image acquisitions. If multiple DSA image acquisitions are used for image reconstruction, individual separately acquired DSA image acquisitions are registered with acquired 3D image volume data and registration adjustments are factored into projection calculations. Image data processor 15 uses 3D image data representing a 3D imaging volume including vessels in determining a transit time curve for an individual volume image element (e.g., a pixel) in a blood vessel. An individual transit time curve identifies imaging luminance content representative values of an individual image element (e.g., a pixel) over a time period. In response to image data processor 15 generating a flow enhanced vascular 3D image using transit time data, the transit time data used in deriving the flow enhanced vascular 3D image utilized is stored as a normal 3D raster image and color map, a 3D polygonal model, or in a proprietary format including the geometry and transit time curve information.

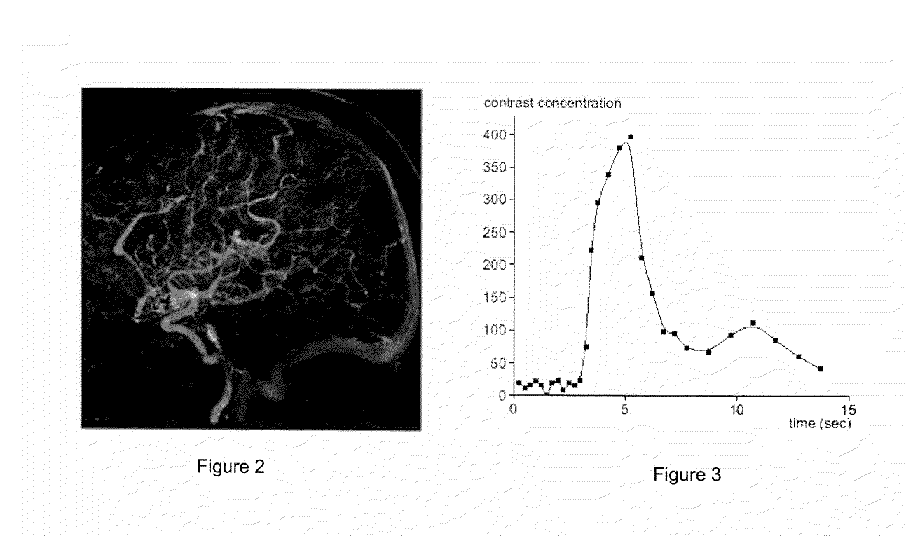

FIG. 2 shows a DSA image presenting a vessel structure (shown in grayscale representing a color coded image). Color or another visual attribute (such as shading, hatching, grayscale, highlighting or other visual indicator) may be used to present blood flow transit time information. In one embodiment, the blood flow transit time information is displayed with varying colors (or other visual attributes) that identify the time at which blood flow achieves a desired characteristic. FIG. 3 shows a transit time curve for one pixel in the DSA image of FIG. 2 identifying imaging luminance content representative values of an individual image element (e.g., a pixel) or groups of elements over a time period.

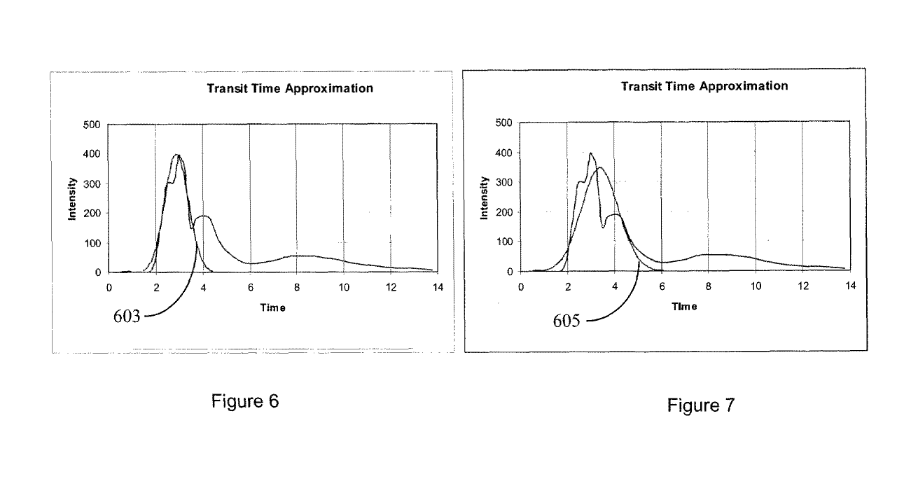

Image data processor 15 computes an initial transit time curve for individual voxels of a 3D imaging volume. This may involve Gaussian modeling of a transit time curve fitting a single Gaussian function to a pixel transit time curve as described later in connection with FIGS. 6-8. FIG. 9 illustrates the X-ray projection of a selected voxel 669 onto x-ray detectors 653 and 657 to determine the pixels in each DSA image that project to the selected voxel 671 and 673. Processor 15 averages the transit time curves of the pixels projecting to the selected voxel 671 and 673 in each plane to produce two averaged transit time curves (one for each plane). Processor 15 combines the two averaged transit time curves to determine the initial transit time curve of the selected voxel 669.

FIG. 9 illustrates the projection line 660 of an individual pixel 675 through the volume 650 from the x-ray detector 657 to the x-ray source 663. Processor 15 sums the transit time curves of the voxels along the projection line 665 to determine the projected transit time curve for the selected pixel 675. Processor 15 compares the projected transit time curve with the transit time curve for the selected pixel 675 and determines a scaling function for the selected pixel 675. These per pixel scaling functions are used by processor 15 to adjust the transit time curves of the voxels in the volume. Processor 15 computes two average scaling functions (one for each DSA image) by averaging scaling functions of the pixels in DSA images that project through selected voxels 671 and 673. Processor 15 computes a voxel scaling function from the average scaling functions. Specifically, the average scaling functions are compared and the highest scaling value is used at each discrete time step. Processor 15 scales the voxel transit time curve by multiplying the voxel transit time curve by the voxel scaling curve. Processor 15 computes per pixel scaling functions and adjusts voxel transit time curves until a completion criterion is met.

Processor 15 manages and expedites these computations by generating a list of voxels comprising part of a vessel in 3D imaging volume 650 and stores data identifying voxel position for each voxel with an intensity value greater than a threshold (indicating the presence of a blood filled vessel). Processor 15 discards or unloads the imaging volume data to free up memory and generates a set of data elements (or pointers to data elements) for the pixels of each 2D DSA image taken through the volume. Processor 15 further: computes initial transit time curves for individual voxels in the list, identifies the per pixel scaling functions for individual pixels, and adjusts the transit time curves for individual voxels in the list. Processor 15 iteratively computes per pixel scaling functions and adjustment of the voxel transit time curves, until a completion criteria is reached. Processor 15 generates new color coded volume data using the transit time curve information to assign colors to the voxels identified in the list.

Processor 15 (FIG. 1) also generates and initializes data elements for these pixels (if the data elements do not exist) comprising sets of time varying data including a projection sum function value and a scaling function value. For individual images, processor 15 computes an averaged transit time curve comprising an average of transit time curves for the pixels projecting to a selected voxel 673 (FIG. 9). The computed average may be a normal average or in another embodiment a center weighted average of the pixels projecting to the selected voxel 673.

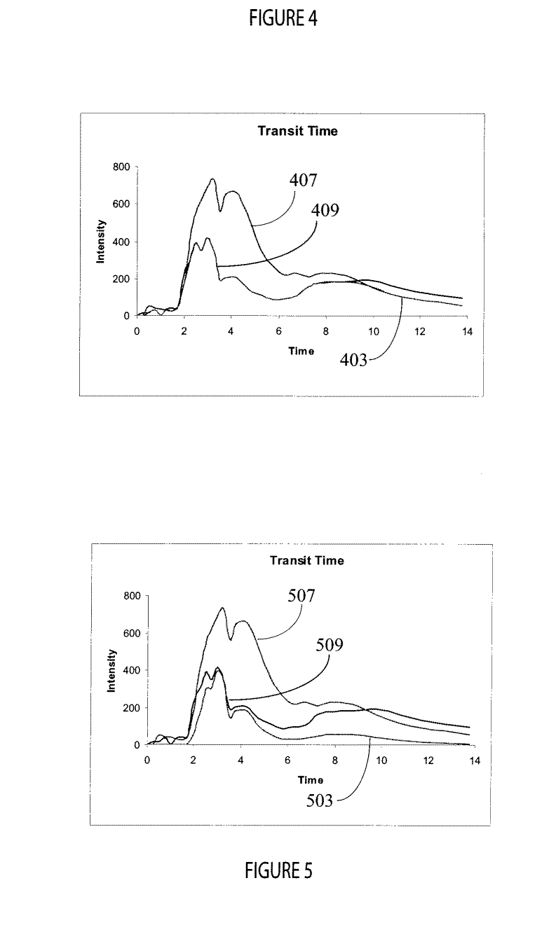

FIG. 4 illustrates generation of a composite single transit time curve by taking minimum luminance intensity values from two different transit time curves obtained by computing the per DSA image average of the transit time curves of the pixels projecting to the selected voxel 671 and 673. Specifically, FIG. 4 illustrates generation of composite single transit time curve 403 derived by processor 15 by taking a minimum luminance intensity value from both an averaged transit time curve 409 for the pixels projecting to the selected voxel 671 in the first DSA image and from an averaged transit time curve 407 for the pixels projecting to the selected voxel 673 in the second DSA image. The transit time curve of corresponding respective individual pixel 675 is derived from the intensity values for that pixel in each frame of the DSA image.

FIG. 5 illustrates generation of a composite single transit time curve by multiplying luminance intensity values from the two different transit time curves obtained by computing the per DSA image average of the transit time curves of the pixels projecting to the selected voxel 671 and 673. Specifically, FIG. 5 illustrates generation of composite single transit time curve 503 derived by processor 15 by multiplying luminance intensity values of an averaged transit time curve 509 derived from the pixels projecting to the selected voxel 671 in the first DSA image with luminance intensity values of an averaged transit time curve 507 derived from the pixels projecting to the selected voxel 673 in the second DSA image.

In one embodiment, processor 15 applies a mask to a transit time curve of a voxel to highlight a region of interest of the transit time curve and reduce influence of the remainder of the curve on further scaling and transit time curve calculations. Processor 15 adds a transit time curve luminance intensity value of a voxel to a sum function value of each pixel involved in the computation of the voxel transit time curve along the projection line. Processor 15 further computes scaling functions for pixels used in this process by dividing a transit time curve by a projection sum function and maintains an overall average scaling function for the pixels processed. The overall average scaling function is the average of the scaling functions for the pixels utilized in the process and is used as an overall indication of the progress of the iterative optimization and is also used to determine when no further iterations are required. Processor 15 re-initializes the projection sum function for each pixel after computing a scaling function and adjusts the transit time curves for each pixel.

For individual images processor 15 generates an average scaling function that is the average of the scaling functions for the pixels projecting to a selected voxel 671 or 673. This may be a direct average or a center weighted average of the scaling functions for the pixels projecting to a selected voxel 671 and 673. Processor 15 computes a voxel scaling function from the average scaling functions. Specifically, the average scaling functions are compared and the highest scaling value is used at each discrete time step. Processor 15 scales the voxel transit time curve by multiplying the voxel transit time curve by the voxel's scaling curve. The steps of generating and applying the scaling function may be iteratively repeated until the overall average scaling function is determined to be acceptable (e.g. to achieve a higher scaling function), or a predetermined number of iterations is reached. The optimum overall averaged scaling function is a horizontal line of value 1.0, indicating that no further scaling is required.

Processor 15 also tracks iteration completion criteria. The iteration completion criteria are a globalized measure of the voxel scaling functions (average, median, mode, maximum). In the case of an optimal embodiment, an acceptable termination criteria may be that the average (or minimum) value of the voxel scaling functions is greater than 0.90, for example. The iteration completion criteria can also have alternate exit criteria (e.g. a maximum number of iterations or time spent iterating).

Processor 15 further stores 3D enhanced vasculature data in a 3D imaging memory and discards or unloads pixel data to free up memory. Processor 15 analyzes transit time curves of voxels (pixels) in the list of voxels to identify the transit time values for voxels comprising a vessel and assigns a zero value to other voxels. Processor 15 analyzes transit time curves to identify characteristics including the time at which blood flow achieves a desired characteristic such as, first detected flow of contrast agent, peak contrast agent enhancement, or maximum gradient (change in rate of blood flow). Display processor 29 displays blood flow transit time characteristics with varying colors (or other visual attributes) on display 19. Other embodiments are used to improve performance or to reduce memory requirements. Specifically, in one embodiment if pixels on projection line 660 produce a summed transit time curve that equals (or is substantially close to) the transit time curve of pixel 675 acquired by X-ray imaging detector 657, the voxels along the projection line 665 are marked as completed and excluded in future iterative processing.

In another embodiment, a 2D color coded image of the vasculature is used to assign colors to a 3D image. The transit time curve for a pixel represents a summation of contrast agent flow through patient anatomy between the pixel and the X-ray source, which means that a transit time curve is not for one vessel but all vessels represented by the pixel. The occurrence of vessel overlap means that processor 15 employs additional logic in selecting a vessel to assign a color in a 2D image, e.g., by differentiating vessels in images in other orientations. Also the voxels for vessels that are not assigned a color need to be assigned a color, which involves identifying the path of the vessel containing the uncolored voxel and assigning color values interpolated from adjacently colored sections of the vessel. The system may combine morphologic and functional information or images into a single image or display for different applications such as combining 3D images and DSA images. The system advantageously displays blood flow information acquired from a DSA acquisition together with vascular morphology obtained by a 3D image acquisition as a single composite combined image.

The functional information for individual 3D image elements (e.g., pixels) is determined by processor 15 by assigning approximated transit time curves of a fundamental shape to each pixel and by making iterative adjustments to these approximated curves. Processor 15 iteratively minimizes a difference between the transit time curves of the pixels in an image acquired by X-ray imaging detector 653 and corresponding calculated (approximated) voxel transit time curves derived along corresponding projection lines (e.g., line 660) to the corresponding pixel 675. A contrast bolus introduced into a vessel is expected to flow through the vessel with a concentration that increases, reaches a maximum value, and decreases over time. In one embodiment, processor 15 models a transit time curve of a voxel as a Gaussian distribution. Other distributions may be employed in alternative embodiments. The presence of an aneurysm or collateral flow may disrupt blood flow dynamics causing the blood to mix, swirl, or flow unevenly, producing an asymmetric curve with multiple peaks. A Gaussian approximation may prove sufficient to model blood flow in the presence of disruptions if it adequately models the portion of the transit time curve of interest (e.g., a location of peak contrast enhancement).

FIGS. 6 and 7 illustrate fitting a Gaussian curve to different portions of a transit time curve. In response to data indicating a desired blood flow characteristic, processor 15 adaptively fits a fundamental (e.g., Gaussian) curve to a portion of a transit time curve derived on projection line 660 to pixel 675. Desired blood flow characteristics include, first detected flow of contrast agent, peak contrast agent enhancement, or maximum gradient (change in rate of blood flow) for example. Processor 15 adaptively selects a fundamental curve type as well as a portion of the transit time curve to be used for fitting to optimize the portion of the curve from which functional blood flow information is extracted. In order to determine peak contrast agent enhancement information, processor 15 may adaptively select a parabolic or Gaussian approximation curve, for example, and localizes the approximation curve (curve 603 shown in FIG. 6) about a peak of the transit time curve. The region of interest is a time interval centered about the peak of the transit time curve.

As illustrated in FIG. 7, processor 15 further adaptively selects and fits a leading edge of Gaussian approximation curve 605 to coincide with the leading edge of a transit time curve for determining information concerning first detected flow of contrast agent and time to the first detected flow. In this case, the region of interest is an interval of time about a location with maximum slope in the transit time curve.

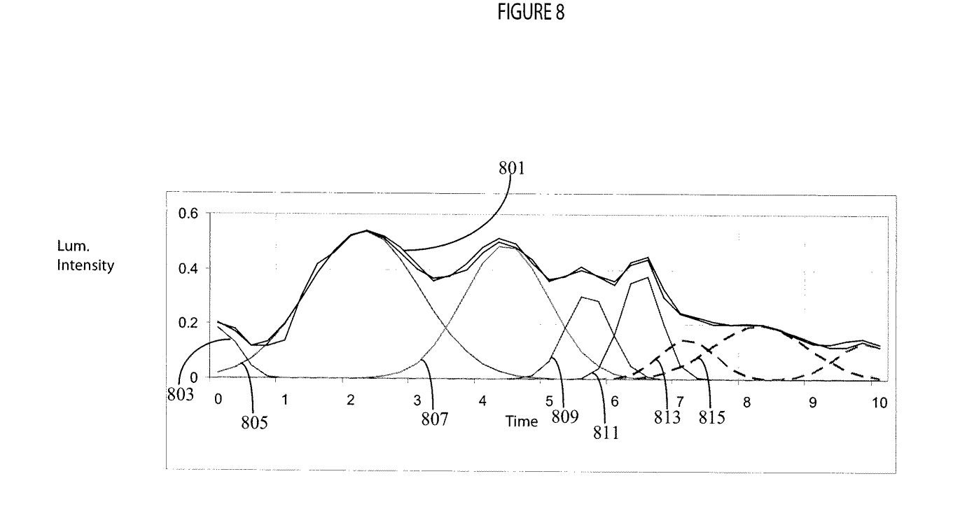

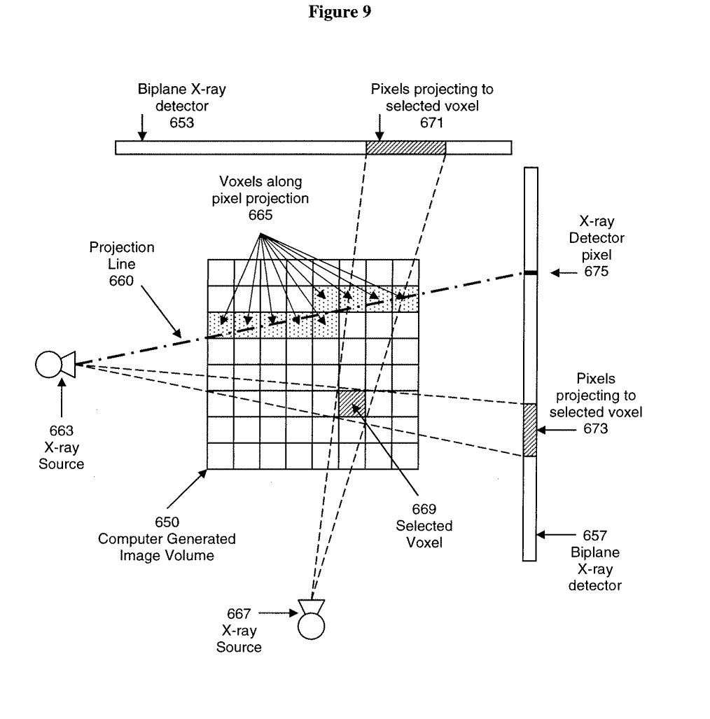

FIG. 8 illustrates employing multiple Gaussian curves to approximate a transit time curve derived on projection line 660 to pixel 675. The projection line represents a path of a single X-ray through the 3D imaging volume. If the transit time curves derived on projection line 660 are properly assigned, the summation of the transit time curves for the individual voxels along the projection line 665 equal the transit time curve acquired by an X-ray detector in a 2D DSA image for that pixel (pixel 675 of FIG. 9). Similarly, the summation of approximated transit time curves of the voxels along a projection line 665 should approximate a transit time curve of the pixel being projected 675. Processor 15 employs Gaussian curves 803, 805, 807, 809, 811, 813, 815 and 817 (FIG. 8) to produce averaged transit time curve 801 of pixel 675.

In one embodiment processor 15 (FIG. 1) generates 3D imaging volume transit time image data by modeling a pixel's projected transit time curve as a Gaussian curve equal to the sum of all of the transit time curve Gaussian approximations for all of the voxels along the pixel projection 665. Processor 15 computes parameter adjustments to the projected transit time curve Gaussian approximation to fit it to the transit time curve of the projected pixel 675. Processor 15 performs these Gaussian curve parameters adjustments iteratively. Processor 15 averages parameter adjustments by dividing individual parameter adjustment elements by an adjustment count and maintains an indication of overall average Gaussian transit time curve parameter adjustments. The averaged parameter adjustments for a pixel are applied by processor 15 to parameters of a Gaussian curve and parameter adjustment elements and adjustment count for the pixel are set to zero. Processor 15 iteratively applies adjustments to Gaussian transit time curve parameters for the voxels along the pixel projection 665 and the adjustments are iteratively derived and applied by repeating the adjustment determination and application steps until overall average Gaussian transit time curve parameter adjustments are acceptable, or an iteration limit is reached.

Processor 15 further generates 3D imaging volume transit time image data comprising enhanced vasculature data by evaluating the generated transit time curves in the list of voxels to identify transit time values for voxels containing a vessel, and assigning a zero value to other voxels. Transit time curves are evaluated to indicate first detected contrast agent, peak contrast agent enhancement, or maximum contrast agent increase.

Processor 15 registers (aligns) the 3D volume imaging data with the generated 2D DSA images and confirms registration is accomplished. In an another embodiment registration is an optional step. If multiple acquired 2D DSA images are used in 3D imaging volume reconstruction, individual acquired 2D DSA images are registered to the 3D imaging volume and adjustments are factored into projection line associated calculations. In response to processor 15 generating 3D volume transit time image data, it is stored as a normal 3D raster image and color map, a 3D polygonal model, or in a proprietary format including geometry and transit time curve information. Processor 15 models a transit time curve of individual voxels using a Gaussian curve, though other different fundamental curve types may also be used. The Gaussian curves are iteratively adjusted to minimize difference between a transit time curve for pixel 675 and the summation of the Gaussian transit time curves for voxels along the pixel projection 665 (FIG. 9).

System 10 employs a clinical workflow in combining 3D medical image data with vessel blood flow information in which a user acquires and reconstructs a 3D image of vascular anatomy of interest. The user acquires a biplane X-ray DSA image of the vascular anatomy and generates a color coded 2D image indicating blood flow characteristics for the acquired biplane DSA image. The user adjusts the color coded image parameters to highlight blood flow characteristics of interest including start time, duration, and type of enhancement (e.g. time to first contrast agent detection or time to peak vessel contrast enhancement). System 10 generates data representing a color coded 3D functional image using the parameters selected for the color coded 2D image and displays the colored 3D image on display 19. The user is able to examine and interact with the 3D functional image by adjusting viewing orientation, start time and duration. In response to a user selecting a position on a vessel in a 3D image presented on display 19, image data processor 15 initiates display of luminance intensity and transit time value for the selected position.

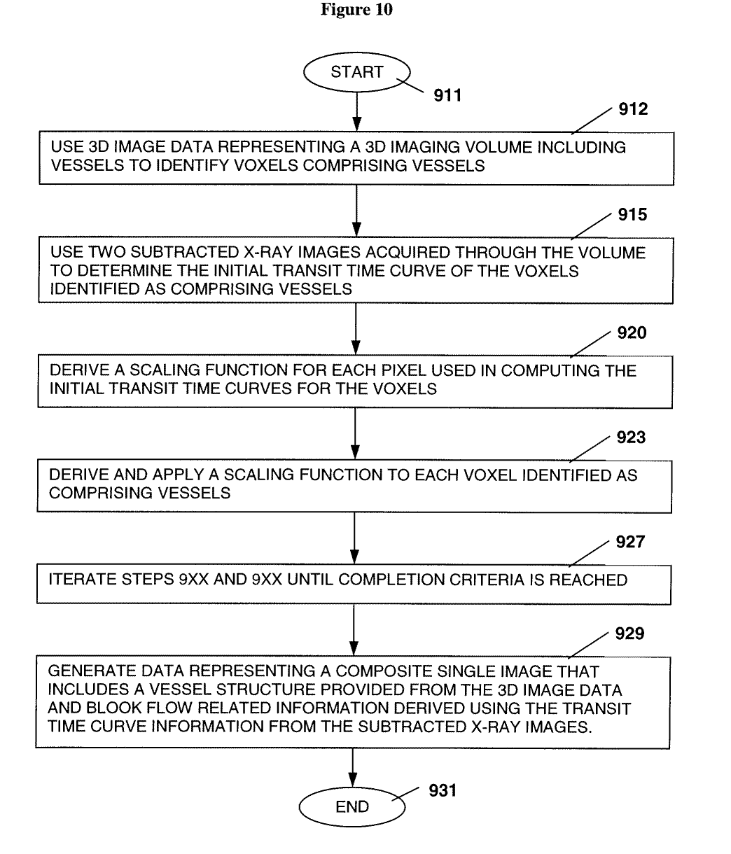

FIG. 10 shows a flowchart of a process used by system 10 (FIG. 1) for combining 3D medical image data with vessel blood flow information. In step 912 following the start at step 911, image data processor 15 uses 3D image data, derived from repository 17, representing a 3D imaging volume including vessels in determining which individual volume image elements (voxels) comprise the vessels and are processed. In step 915 Image data processor 15 computes an initial transit time curve for the voxels identified in step 912 using two or more DSA images that are acquired of the same anatomy as the 3D volume. The 3D image data is provided by at least one of, (a) a rotational X-ray imaging system, (b) a CT scan system, (c) an MRI system and (d) an Ultrasound system. Image data processor 15 in step 915 uses 2D image data, derived from repository 17, representing an X-ray image through the imaging volume in determining a luminance content representative distribution (e.g. a transit time curve) for the individual voxel comprising the vessels in the imaging volume. The 2D X-ray image comprises an image provided by Digital Subtraction Angiography by subtraction of mask image data representing background information from an Angiography image in the presence of a contrast agent, to emphasize vessel structure. An individual transit time curve identifies imaging luminance content representative values of an individual image element over a time period. Image data processor 15 determines the transit time curve for the individual voxel comprising the vessels in the 3D imaging volume by: averaging the transit time curves of the individual pixels in each DSA image that project through the individual voxel and combining these per DSA image averaged transit time curves. In one embodiment, the transit time curves are represented by at least one approximating function comprising a Gaussian distribution representing a transit time curve with a mean value, standard deviation value and amplitude value. Further, image data processor 15 provides multiple compensated transit time curves for corresponding multiple individual image elements comprising the vessels using 2D image data representing multiple X-ray images through the 3D imaging volume. The multiple individual image elements comprising the vessels are pixels and the multiple X-ray images through the 3D imaging volume comprise two or more images having planes intersecting with an angle of separation derived by a biplane X-ray imaging system, for example.

In step 920 processor 15 computes scaling functions for each pixel using the pixel projected transit time curve and transit time curve. The projected transit time curve is the sum of the transit time curve for the voxels in the 3D image that are crossed by the line connecting the pixel on the X-ray detector and the X-ray source. Specifically, processor 15 computes the pixel's projected transit time curve and uses it to create a scaling function for each pixel in addition to the pixel's transit time curve. In step 923 pixel scaling functions are used to derive and apply voxel scaling functions to the transit time curves of the voxels comprising the vessels. In step 927 processor 15 evaluates information collected concerning scaling functions calculated to determine if completion criteria has been reached. If the completion criteria have not been met, processor 15 repeats steps 920 and 923 until the completion criteria is satisfied. In step 929, display processor 29 provides data representing a composite single displayed image comprising a vessel structure including blood flow related information derived using the compensated transit time curves and the 3D image data. In one embodiment, the volume image element is a voxel and the derived blood flow related information is derived using the compensated luminance content representative distribution and the 3D image data. The process of FIG. 10 terminates at step 931.

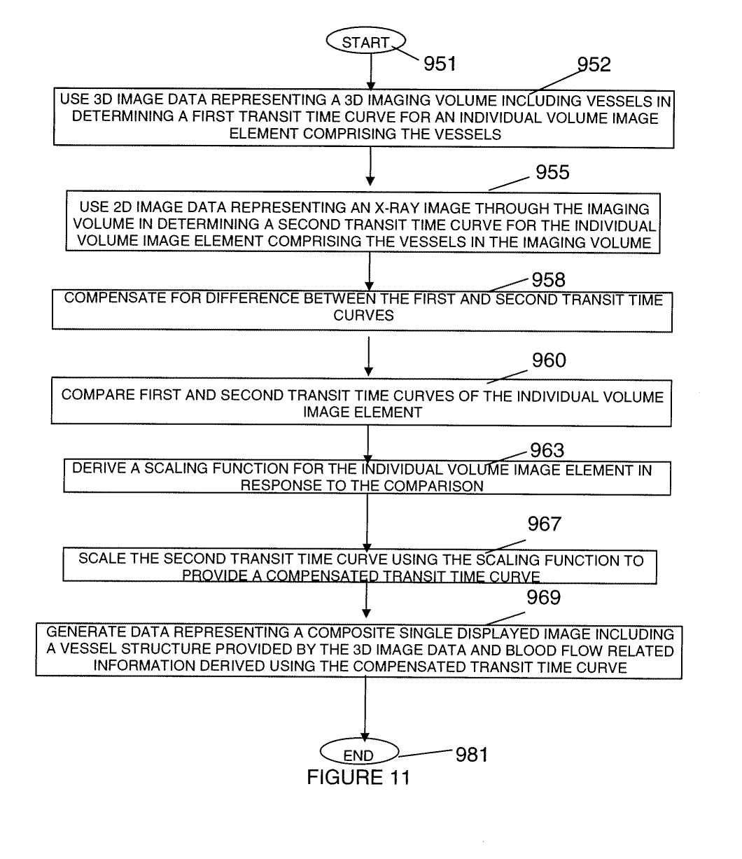

FIG. 11 shows a flowchart of another process embodiment used by system 10 (FIG. 1) for combining 3D medical image data with vessel blood flow information. In step 952 following the start at step 951, image data processor 15 uses 3D image data, derived from repository 17, representing a 3D imaging volume including vessels in determining a first luminance content representative distribution (e.g., a first transit time curve in the presence of a contrast agent) for an individual volume image element comprising the vessels. An individual transit time curve identifies imaging luminance content representative values of an individual image element over a time period. The 3D image data is provided by at least one of, (a) a rotational X-ray imaging system, (b) a CT scan system, (c) an MRI system and (d) an Ultrasound system. Image data processor 15 in step 955 uses 2D image data, derived from repository 17, representing an X-ray image through the imaging volume in determining a second luminance content representative distribution (e.g. a transit time curve) for the individual volume image element comprising the vessels in the imaging volume. The 2D X-ray image comprises an image provided by Digital Subtraction Angiography by subtraction of mask image data representing background information from an Angiography image in the presence of a contrast agent, to emphasize vessel structure. Image data processor 15 determines the second transit time curve for the individual volume image element comprising the vessels in the 3D imaging volume by summing transit time curves of individual pixels along a linear path (projection line) through a 2D X-ray image.

Image data processor 15 uses the 3D image data and the 2D image data in deriving blood flow related information for the vessels by determining and comparing luminance content representative values of an individual volume image element in the vessels in the imaging volume over a time period, in the presence of a contrast agent. Specifically, processor 15 processes the second luminance content representative distribution (second transit time curve) to compensate for difference between the first and second distributions (transit time curves) to provide a compensated distribution (transit time curve). In one embodiment, the luminance content representative distributions are represented by at least one approximating function comprising a Gaussian distribution representing a luminance content representative distribution with a mean value, standard deviation value and amplitude value. Further, image data processor 15 provides multiple compensated transit time curves for corresponding multiple individual image elements comprising the vessels using 2D image data representing multiple X-ray images through the 3D imaging volume. The multiple individual image elements comprising the vessels are pixels and the multiple X-ray images through the 3D imaging volume comprise two or more images having planes intersecting with an angle of separation derived by a biplane X-ray imaging system, for example.

In step 958 processor 15 compensates for difference between the first and second transit time curves by, in step 960 comparing first and second transit time curves of the individual volume image element, in step 963 deriving a scaling function for the individual volume image element in response to the comparison and in step 967 scaling the second transit time curve using the scaling function to provide a compensated transit time curve. In step 969, display processor 29 provides data representing a composite single displayed image comprising a vessel structure including blood flow related information derived using the compensated transit time curves and the 3D image data. In one embodiment, the volume image element is a voxel and the derived blood flow related information is derived using the compensated luminance content representative distribution and the 3D image data The process of FIG. 10 terminates at step 981.

A pixel comprises one or more image elements in a 2D image and a voxel comprises one or more image elements in a 3D imaging volume. The terms pixel and voxel are used interchangeably herein as 2D images are encompassed within a 3D imaging volume and hence a pixel is typically the same as a voxel. A processor as used herein is a device for executing machine-readable instructions stored on a computer readable medium, for performing tasks and may comprise any one or combination of, hardware and firmware. A processor may also comprise memory storing machine-readable instructions executable for performing tasks. A processor acts upon information by manipulating, analyzing, modifying, converting or transmitting information for use by an executable procedure or an information device, and/or by routing the information to an output device. A processor may use or comprise the capabilities of a controller or microprocessor, for example, and is conditioned using executable instructions to perform special purpose functions not performed by a general purpose computer. A processor may be coupled (electrically and/or as comprising executable components) with any other processor enabling interaction and/or communication there-between. A user interface processor or generator is a known element comprising electronic circuitry or software or a combination of both for generating display images or portions thereof. A user interface comprises one or more display images enabling user interaction with a processor or other device.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters. A graphical user interface (GUI), as used herein, comprises one or more display images, generated by a display processor and enabling user interaction with a processor or other device and associated data acquisition and processing functions.

The UI also includes an executable procedure or executable application. The executable procedure or executable application conditions the display processor to generate signals representing the UI display images. These signals are supplied to a display device which displays the image for viewing by the user. The executable procedure or executable application further receives signals from user input devices, such as a keyboard, mouse, light pen, touch screen or any other means allowing a user to provide data to a processor. The processor, under control of an executable procedure or executable application, manipulates the UI display images in response to signals received from the input devices. In this way, the user interacts with the display image using the input devices, enabling user interaction with the processor or other device. The functions and process steps (e.g., of FIG. 8) herein may be performed automatically or wholly or partially in response to user command. An activity (including a step) performed automatically is performed in response to executable instruction or device operation without user direct initiation of the activity. Workflow comprises a sequence of tasks performed by a device or worker or both. An object or data object comprises a grouping of data, executable instructions or a combination of both or an executable procedure.

The system and processes of FIGS. 1-10 are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. The system generates 3D imaging volume blood flow transit time data comprising enhanced vasculature data indicating blood flow characteristics. Further, the processes and applications may, in alternative embodiments, be located on one or more (e.g., distributed) processing devices on the network of FIG. 1. Any of the functions and steps provided in FIGS. 1-10 may be implemented in hardware, software or a combination of both.

What is claimed is:

1. A system for combining 3D medical image data with vessel blood flow information, comprising:
at least one repository for storing,
3D image data representing a 3D imaging volume including vessels in the presence of a contrast agent and
2D image data representing a 2D X-ray image through said imaging volume in the presence of a contrast agent;
an image data processor for using said 3D image data and said 2D image data in deriving blood flow related information for said vessels; and
a display processor for providing data representing a composite single displayed image including a vessel structure provided by the 3D image data and the derived blood flow related information.

2. A system according to claim 1, wherein
said image data processor derives said blood flow related information for said vessels by determining and comparing luminance content representative values of an individual volume image element in said vessels in said imaging volume over a time period, in the presence of a contrast agent.

3. A system according to claim 1, wherein
said 3D image data represents a 3D imaging volume including vessels produced in the presence of a contrast agent.

4. A system according to claim 2, wherein
said image data processor determines and compares luminance content representative values of said individual volume image element by
using said 3D image data in determining a first luminance content representative distribution for said individual volume image element comprising said vessels, an individual luminance content representative distribution identifying imaging luminance content representative values of an individual image element over a time period, in the presence of a contrast agent,
using 2D image data representing an X-ray image through said imaging volume in determining a second luminance content representative distribution for said individual volume image element comprising said vessels in said imaging volume and
processing said second luminance content representative distribution to compensate for difference between said first and second luminance content representative distributions to provide a compensated luminance content representative distribution.

5. A system according to claim 4, wherein
the luminance content representative distributions are represented by at least one approximating function.

6. A system according to claim 5, wherein
the approximating function is a Gaussian distribution representing a luminance content representative distribution with a mean value, standard deviation value and amplitude value.

7. A system according to claim 4, wherein
said volume image element is a voxel and
said derived blood flow related information is derived using said compensated luminance content representative distribution and said 3D image data

8. A system according to claim 1, wherein
said 2D X-ray image comprises an image provided by Digital Subtraction Angiography by subtraction of mask image data representing background information from an Angiography image, to emphasize vessel structure.

9. A system according to claim 1, wherein
said 3D image data is provided by at least one of, (a) a rotational X-ray imaging system, (b) a CT scan system, (c) an MRI system and (d) an Ultrasound system.

10. A system for combining 3D medical image data with vessel blood flow information, comprising:
an image data processor for,
using 3D image data representing a 3D imaging volume including vessels in determining a first transit time curve for an individual volume image element comprising said vessels, an individual transit time curve identifying imaging luminance content representative values of an individual image element over a time period,
using 2D image data representing at least an X-ray image through said imaging volume in determining a second transit time curve for said individual volume image element comprising said vessels in said imaging volume and
processing said second transit time curve to compensate for difference between said first and second transit time curves to provide a compensated transit time curve; and
a display processor for providing data representing a single composite displayed image comprising a vessel structure including blood flow related information derived using said compensated transit time curve and said 3D image data.

11. A system according to claim 10, wherein
said image data processor determines said second transit time curve for said individual volume image element comprising said vessels in said 3D imaging volume by summing transit time curves of individual pixels along a linear path through a 2D X-ray image.

12. A system for combining 3D medical image data with vessel blood flow information, comprising:
an image data processor for,
using 3D image data representing a 3D imaging volume including vessels in determining a first transit time curve for an individual volume image element comprising said vessels, an individual transit time curve identifying imaging luminance content representative values of an individual image element over a time period,
using 2D image data representing an X-ray image through said imaging volume in determining a second transit time curve for said individual volume image element comprising said vessels in said imaging volume and
compensating for difference between said first and second transit time curves by,
comparing first and second transit time curves of said individual volume image element,
deriving a scaling function for said individual volume image element in response to the comparison and
scaling said second transit time curve using said scaling function to provide a compensated transit time curve; and
a display processor for providing data representing a composite single displayed image including a vessel structure provided by the 3D image data and blood flow related information derived using said compensated transit time curve.

13. A system according to claim 12, wherein
said image data processor provides a plurality of compensated transit time curves for a corresponding plurality of individual image elements comprising said vessels using 2D image data representing a plurality of X-ray images through said 3D imaging volume.

14. A system according to claim 13, wherein
said plurality of individual image elements comprising said vessels are pixels.

15. A system according to claim 13, wherein
said plurality of X-ray images through said 3D imaging volume comprise two or more images having planes intersecting with an angle of separation.

16. A computer implemented method for combining 3D medical image data with vessel blood flow information, comprising the activities of:
storing in at least one repository for,
3D image data representing a 3D imaging volume including vessels in the presence of a contrast agent and
2D image data representing a 2D X-ray image through said imaging volume in the presence of a contrast agent;
employing said 3D image data and said 2D image data in deriving blood flow related information for said vessels; and
generating data representing a composite single displayed image including a vessel structure provided by the 3D image data and the derived blood flow related information.
